(19) United States
(12) Patent Application Publication
(10) Pub. No.: US 2026/0345007 A1
(43) Pub. Date: Nov. 5, 2026

(54) COMBINATION OF DYNAMIC AND STATIC FORCES IN EVALUATION OF ERGONOMICS RISK FACTORS FOR TENDON INJURY

(71) Applicant: The Boeing Company, Arlington, VA (US)

(72) Inventors: Kevin F. MALIK, Seattle, WA (US); Karen C. TAKATANI, Renton, WA (US); Liisa Charlie HAMMER, Seattle, WA (US)

(73) Assignee: The Boeing Company, Arlington, VA (US)

(21) Appl. No.: 19/197,144

(22) Filed: May 2, 2025

(51) Int. Cl. A61B 5/00

(52) U.S. Cl. CPC A61B 5/0051; A61B 5/4884; A61B 5/7275; A61B 2503/20; A61B 2562/0219

(57) ABSTRACT

A method to determine failure metrics for target tissue by characterizing surrounding tissues as a damper, determining a tool and a risk factor associated with a task, collecting data from accelerometers or other measurement devices that are monitoring behavior of the target and surrounding tissue while the tool is being used to perform the task, determining an effective damping coefficient by comparing amplitude and frequency distributions of the collected data, providing the effective damping coefficient to a finite element model, predicting and modeling effective resultant forces, frequencies, and amplitudes at a first pre-selected location in the tissue, determining an FEM geometry for the first pre-selected location in the tissue, determining material properties for the tissue, determining boundary conditions and static forces before use of the tool, providing the collected data to the FEM, and determining the failure metrics.

### FIELD OF THE DISCLOSURE

The present disclosure is directed to predicting failure metrics in performing a task.

### BACKGROUND

Overuse injuries, particularly of the shoulder, including the supraspinatus tendon, are some of the most significant of ergonomics injuries. Accordingly, there is a need to understand the dynamics of workplace or other activities that contribute to and can result in stress, for example to a tendon, to address a task or repetition of tasks that contribute to an injury. Ergonomic models that rely on observation of the human body at work, estimates of forces using psychosocial measurements, sensor measurements, such as, for example, but not limited to, accelerometer or other measurement device measurements of a vibrating tool, and comparing these to three distinct guideline tables to identify risk for posture, force and vibration, independently have been used. In some ergonomics and/or biomechanics applications, posture, force and vibration are treated as separate risks with their own risk thresholds for duration or repetition.

Published tables of risk created by government or industry groups for ergonomic factors, with the results threshold listed as low, medium or high risk, are available. Chart data are based on, for example, using a vibrating tool, and the amount of vibration and the duration of the exposure are used to determine if the exposure is low, medium, or high. The chart data do not take into account that the tool is being used, for example, near the waist with elbows bent, or stretched overhead, where strength and stamina is typically lower. Nor do the data take into account that the tool is, for example, a small, powered screwdriver that requires almost no push force, or a jackhammer which requires nearly full body weight to control. The thresholds low, medium, or high are based on general injury statistics and they are used to measure the improvement when moving from high to medium or medium or low, in order to improve the situation. This approach does not correspond to a numerical probability of injury.

What is needed is a way to quantify tissue failure based on data collected during use of a tool. What is needed is a characterization of the tissue involved in the use of the tool. The characterization can include modeling the tissue as, for example, but not limited to, a damper, a simple machine, or other analogous mechanism representative of the specific task being modeled. What is needed is a failure metric to assess when the tissue will fail under specific circumstances. What is needed is a tool metric to compare the failure metrics of various tools.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a method operable to determine failure metrics for target tissue exposed to vibration, using inputs of surrounding tissue, which are characterized as a system of mechanisms representative of a task. The method includes (a) determining a tool and a risk factor associated with the task, (b) collecting data from sensors that are monitoring behavior of the target tissue and the surrounding tissue while the tool is being used to perform the task, the sensors being positioned at pre-selected nodes and insertions in proximity to the target tissue and the surrounding tissue, (c) repeating (a) and (b) for a plurality of tools, and a plurality of risk factors, (d) determining a representative metric by comparing amplitude and frequency distributions of the collected data at the target tissue and the surrounding tissue, (e) providing the representative metric to a finite element model (FEM), (f) predicting and modeling effective resultant forces, frequencies, and amplitudes at a first location in the target tissue, for an applied force, vibration frequencies, and amplitudes experienced at a second location in the surrounding tissue for the tool and the risk factor, (g) determining an FEM geometry for the first location in the target tissue based on the effective resultant forces, the vibration frequencies, and the amplitudes at the first location in the target tissue, (h) determining material properties for the target tissue and the surrounding tissue, (i) determining boundary conditions and static forces before use of the tool, (j) providing the collected data, the FEM geometry, the material properties, the boundary conditions, and the static forces to the FEM, and (k) determining, by executing the FEM, the failure metrics when the first location in the target tissue will fail for the plurality of the risk factors and the plurality of the tools. Other embodiments of this aspect include corresponding computer systems, apparatus, and computer programs recorded on one or more computer storage devices, each configured to perform the actions of the methods.

One general aspect includes a computer system operable to determine failure metrics for target tissue exposed to vibration, using inputs of surrounding tissue, which are characterized as a system of mechanisms representative of a task. The computer system includes a hardware processor, and a non-volatile storage medium storing instructions that when executed by the hardware processor perform operations. The instructions may include (a) determining a tool and a risk factor associated with the task, (b) collecting data from sensors that are monitoring behavior of the target tissue and the surrounding tissue while the tool is being used to perform the task, the sensors being positioned at pre-selected nodes and insertions in proximity to the target tissue and the surrounding tissue, (c) repeating (a) and (b) for a plurality of tools, and a plurality of risk factors, (d) determining a representative metric by comparing amplitude and frequency distributions of the collected data at the target tissue and the surrounding tissue, (e) providing the representative metric to a FEM, (f) predicting and modeling effective resultant forces, frequencies, and amplitudes at a first location in the target tissue, for an applied force, vibration frequencies, and amplitudes experienced at a second location in the surrounding tissue for the tool and the risk factor, (g) determining an FEM geometry for the first location in the target tissue based on the effective resultant forces, the vibration frequencies, and the amplitudes at the first location in the target tissue, (h) determining material properties for the target tissue and the surrounding tissue, (i) determining boundary conditions and static forces before use of the tool, (j) providing the collected data, the FEM geometry, the material properties, the boundary conditions, and the static forces to the FEM, and (k) determining, by executing the FEM, the failure metrics when the first location in the target tissue will fail for the plurality of the risk factors and the plurality of the tools. Other embodiments of this aspect include corresponding computer systems, apparatus, and computer programs recorded on one or more computer storage devices, each configured to perform the actions of the methods.

One general aspect includes a computer program product operable to determine failure metrics for target tissue exposed to vibration, using inputs of surrounding tissue, which are characterized as a system of mechanisms representative of a task. The computer program product also includes (a) determining a tool and a risk factor associated with the task, (b) collecting data from sensors that are monitoring behavior of the target tissue and the surrounding tissue while the tool is being used to perform the task, the sensors being positioned at pre-selected nodes and insertions in proximity to the target tissue and the surrounding tissue, (c) repeating (a) and (b) for a plurality of tools, and a plurality of risk factors, (d) determining a representative metric by comparing amplitude and frequency distributions of the collected data at the target tissue and the surrounding tissue, (e) providing the representative metric to a FEM, (f) predicting and modeling effective resultant forces, frequencies, and amplitudes at a first location in the target tissue, for an applied force, vibration frequencies, and amplitudes experienced at a second location in the surrounding tissue for the tool and the risk factor, (g) determining an FEM geometry for the first location in the target tissue based on the effective resultant forces, the vibration frequencies, and the amplitudes at the first location in the target tissue, (h) determining material properties for the target tissue and the surrounding tissue, (i) determining boundary conditions and static forces before use of the tool, (j) providing the collected data, the FEM geometry, the material properties, the boundary conditions, and the static forces to the FEM, and (k) determining, by executing the FEM, the failure metrics when the first location in the target tissue will fail for the plurality of the risk factors and the plurality of the tools. Other embodiments of this aspect include corresponding computer systems, apparatus, and computer programs recorded on one or more computer storage devices, each configured to perform the actions of the methods.

It is to be understood that both the foregoing general description and the following detailed description are exemplary and explanatory only and are not restrictive of the present teachings, as claimed.

### BRIEF DESCRIPTION OF THE DRAWINGS

The accompanying drawings, which are incorporated in and constitute a part of this specification, illustrate aspects of the present teachings and together with the description, serve to explain the principles of the present teachings.

FIG. 1A is a photographic illustration of a subject outfitted with accelerometers in accordance with embodiments of the present disclosure;

FIG. 1B is a photographic illustration of a subject operating a tool while outfitted with accelerometers in accordance with embodiments of the present disclosure;

FIG. 2 is a pictorial illustration of accelerometer data gathered from the subject of FIGS. 1A and 1B;

FIGS. 3A and 3B are flowcharts of a method in accordance with embodiments of the present disclosure; and

FIG. 4 is a schematic block diagram of a system in accordance with embodiments of the present disclosure.

It should be noted that some details of the figures have been simplified and are drawn to facilitate understanding rather than to maintain strict structural accuracy, detail, and scale.

### DESCRIPTION

Reference will now be made in detail to the present teachings, examples of which are illustrated in the accompanying drawings. In the drawings, like reference numerals have been used throughout to designate identical elements. In the following description, reference is made to the accompanying drawings that form a part thereof, and in which is shown by way of illustration specific examples of practicing the present teachings. The following description is, therefore, merely exemplary.

Vibration effects can be characterized from, for example, the hand, through the body, and into the soft tissue. Characterizing soft tissue material behavior by capturing the viscoelastic behavior across systems, and characterizing the effects of, for example hand/arm/limb (and other parts) vibration, can highlight accelerating material fatigue throughout the soft tissue geometry and accelerating injury. Specifically, body parts can be modeled as one or a combination of well-known devices, and the effects of repetitive use of industrial tools on the body parts can be evaluated to create tissue failure metrics and tool comparisons.

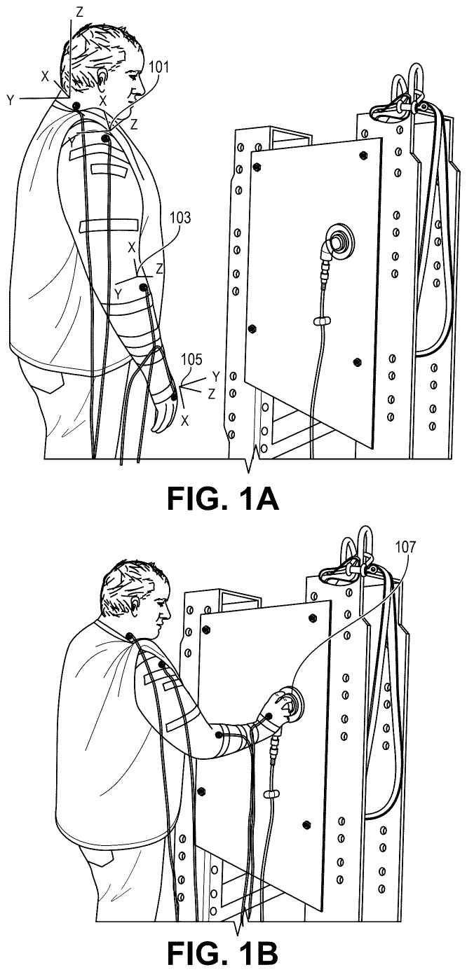

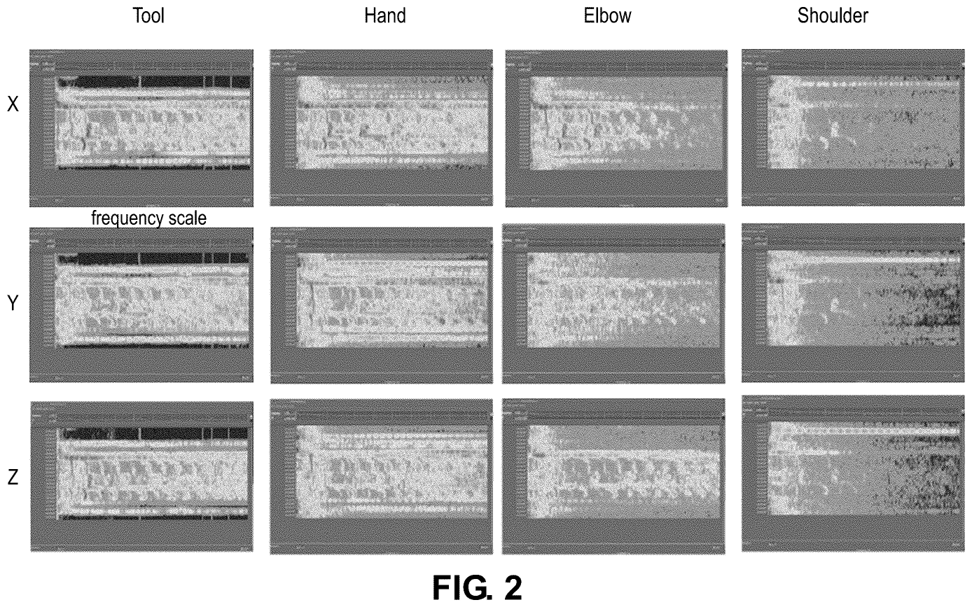

Referring now to FIGS. 1A and 1B, a human body part can be modeled as a device whose characteristics are understood by a FEM. In some configurations, an arm can be modeled as a damper for the purpose of using finite element modeling to determine a failure situation with respect to the use of equipment by the arm. In some configurations, collecting data to support the arm's characterization as a damper can include placing accelerometers at joints (or nodes) in the arm, for example, a shoulder accelerometer 101 (FIG. 1A), an elbow accelerometer 103 (FIG. 1A), and a wrist accelerometer 105 (FIG. 1A). A user so equipped operates the equipment 107 (FIG. 1B) while accelerometer data are gathered. The simultaneous data can be gathered for multiple subjects, tools, and posture variations to enable statistical confidence.

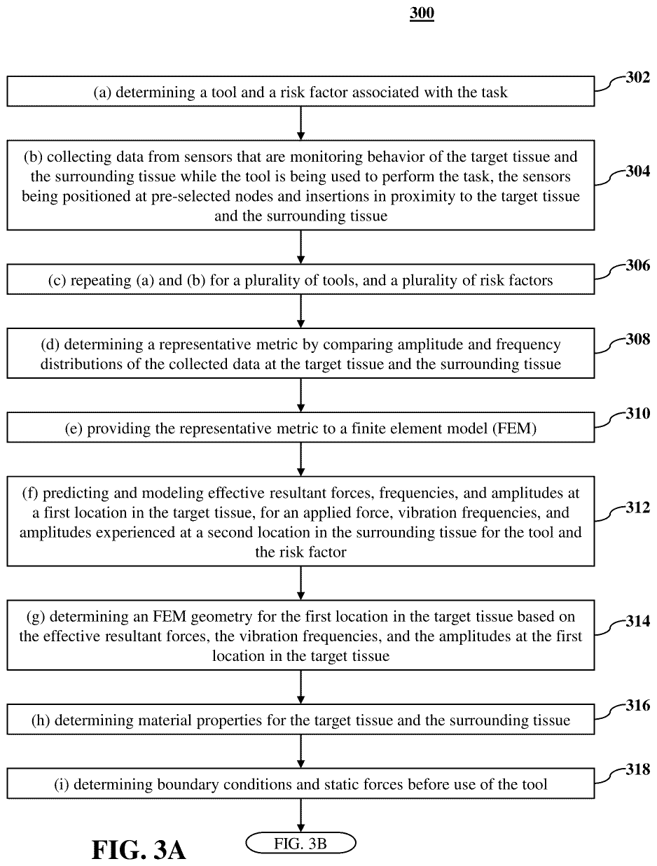

Referring now to FIG. 2, a display of amplitude and frequency as a function of time for the accelerometer locations as shown in FIGS. 1A and 1B is shown.

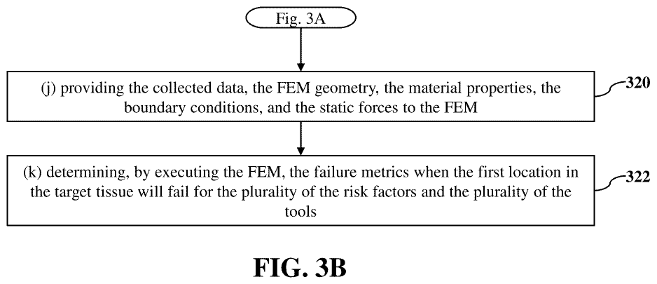

Referring now to FIGS. 3A and 3B, a method 300 for determining failure metrics by taking advantage of the tissue characterization as described herein can include, but is not limited to including, determining 302 tools and postures associated with workplace tasks of interest, and outfitting test subjects with 3D accelerometers at nodes and at tendon insertion points. The method 300 can include gathering 304 simultaneous data while the task is performed, and interpreting raw accelerometer data through spectrogram outputs. The method 300 can include repeating 306 blocks 302 and 304 for a variety of tools, subjects, and risk factors, and determining 308 an effective damping coefficient by comparing the amplitudes and frequency distributions for the accelerometer locations. The method 300 can include providing 310 the effective damping coefficient as input to a FEM and predicting 312 effective resulting forces, frequencies, and amplitudes at a first body location for a given applied force, vibration frequencies, and amplitudes experienced at a second body location for a given tool type and posture. For example, the first body location can be a shoulder, for example, the shoulder Supraspinatus Tendon (SST).

The method 300 can include establishing 314 or importing 3D FEM geometry for a body location, choosing a mesh size based on desired computational accuracy, desired processing load, and desired analysis time. For example, the FEM geometry for the SST and hand-arm-shoulder complex can be established or imported.

The method 300 can include populating 316 material properties for human tissue such as, for example, but not limited to, tendon, muscle, and bone. Material properties can include, but are not limited to including, isotropic and anisotropic modulus values, Poisson's Ratio, and nonlinear, frequency dependent, viscoelastic, and orthotropic parameters.

The method 300 can include establishing 318 model boundary conditions and static forces as a baseline condition prior to application of vibrating hand tool inputs.

The method 300 can include applying 320 data gathered as described with respect to FIGS. 1A and 1B to the FEM to replicate the desired posture and a tool. The data from the collectors are provided at the same time to the FEM. This is done for different tools and postures. In some configurations, a sample size is 24-100 subjects. The data can be used to create the failure metrics by compiling the outputs, comparing different tasks, taking a simple difference, tweaking variables related to posture, determining when tendons will fail, computing the relative difference, and selecting a task with higher failure regime.

The method 300 can also include determining 322 failure characteristics by executing the FEM. The failure characteristics include, but aren't limited to including, time or cycles to failure at the body location for a specific posture and tool application. The method can include repeating executing the FEM for various postures and tool applications to generate predictive outputs for different configurations. The method can include compiling the predictive outputs into a format that can be used for reference for general applications. The format can include, for example, various forms of lookup tables, or other data storage and retrieval mechanisms.

The method can also include compiling a vibrating tool metric based on the generated parameterized solution space for a wide range of tasks and postural variations. The vibrating tool metric can, for example, isolate the contribution of dynamic tool vibration to the damage model, separated from the static contributions of posture and force. The method can also include compiling a tool comparison metric by comparing failure predictions for various tools utilized in similar tasks and postures. In some configurations, the tool comparison metric can be used to evaluate and compare tools for risk of injury. With respect to the tool comparison metric, handheld tools vibrate, and the vibration emitted from the tool can affect a first body location and adjacent body locations. For example, vibration felt at the hand can also be experienced in the arm and shoulder. The vibration can cause capillaries to constrict and can weaken the collagen structure to the tendon, contributing to injuries. Some vibration frequencies are damped with the tool. During some tool use, there is a spike in vibration, and a start-up impulse.

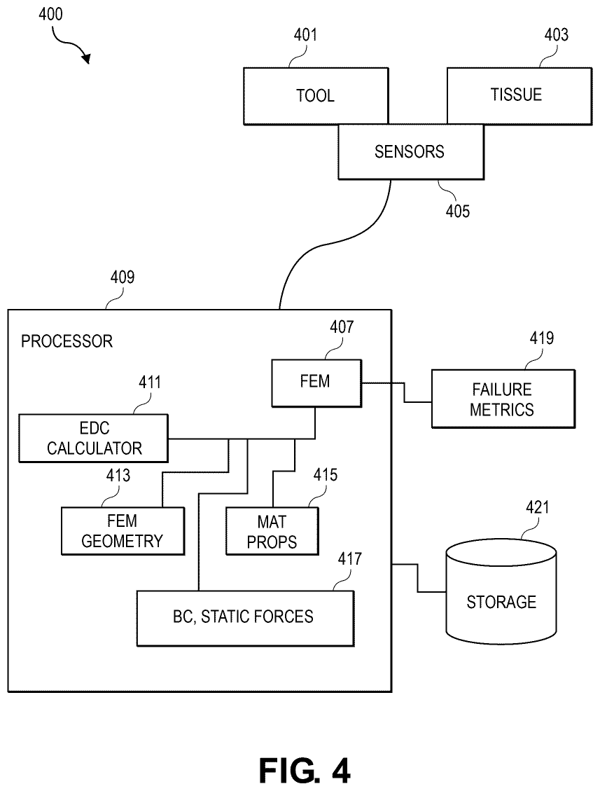

Referring now to FIG. 4, a computer system 400 operable to determine failure metrics for target tissue exposed to vibration, using inputs of surrounding tissue, which are characterized as a system of mechanisms representative of a task includes a hardware processor 409 and a non-volatile storage medium 421 storing instructions that when executed by the hardware processor 409 perform operations. The system 400 can include sensors 405 sensing characteristics of a tool 401 and tissue 403 associated with the task. The tissue 403 includes a target tissue and surrounding tissue in proximity to the target tissue. The target tissue is locate in proximity to the tool, while the surrounding tissue experiences the effects of the tool's motion. In some configurations, the characteristics are risk factors associated with the use of the tool 401 in proximity to the tissue 403. The sensors 405 provide data to the processor 409 as they monitor the behavior of the tissue 403 while the tool 401 is being used to perform the task. The sensors 405 are positioned at pre-selected nodes and insertions in proximity to the tissue 403. The sensors 405 can collect data and provide those data to the processor 409 for a plurality of samples of the tissue 403, possibly from various subjects, a plurality of the tools 401, and a plurality of risk factors. In some configurations, an effective damping coefficient calculator 411 determines an effective damping coefficient by comparing amplitude and frequency distributions of the collected sensor data, and provides the effective damping coefficient to the FEM. In some configurations, the effective damping coefficient calculator 411 can predict and model effective resultant forces, frequencies, and amplitudes at a first pre-selected location in the tissue, for a pre-selected applied force, vibration frequencies, and amplitudes experienced at a second pre-selected location in the tissue for a type of the tool and a risk factor. In some configurations, an FEM geometry calculator 413 determines an FEM geometry for the first location in the tissue 403 based on the effective resultant forces, the vibration frequencies, and amplitudes at the first location in the target tissue 403. In some configurations, a material properties calculator 415 determines material properties for the tissue 403. In some configurations, a boundary conditions and static forces calculator 417 determines boundary conditions and static forces before use of the tool 401. The collected data, the FEM geometry, the material properties, the boundary conditions, and the static forces are provided to the FEM 407. The FEM 407 determines, by executing the FEM, the failure metrics 419 when the first location in the tissue 403 will fail for a plurality of the risk factors and a plurality of the tools 401.

The system 400 can be used to model other mechanisms in addition to a damper such as, for example, simple machines or mechanism representative of a specific task. Thus, the effective damping calculator 411 could be another type of calculator associated with the mechanism being modeled.

While the present teachings have been illustrated with respect to one or more implementations, alterations and/or modifications can be made to the illustrated examples without departing from the spirit and scope of the appended claims. In addition, while a particular feature of the present teachings may have been disclosed with respect to only one of several implementations, such feature may be combined with one or more other features of the other implementations as may be desired and advantageous for any given or particular function. As used herein, the terms “a”, “an”, and “the” may refer to one or more elements or parts of elements. As used herein, the terms “first” and “second” may refer to two different elements or parts of elements. As used herein, the term “at least one of A and B” with respect to a listing of items such as, for example, A and B, means A alone, B alone, or A and B. Those skilled in the art will recognize that these and other variations are possible. Furthermore, to the extent that the terms “including,” “includes,” “having,” “has,” “with,” or variants thereof are used in either the detailed description and the claims, such terms are intended to be inclusive in a manner similar to the term “comprising.” Further, in the discussion and claims herein, the term “about” indicates that the value listed may be somewhat altered, as long as the alteration does not result in nonconformance of the process or structure to the intended purpose described herein. Finally, “exemplary” indicates the description is used as an example, rather than implying that it is an ideal.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompasses by the following claims.

1. A method operable to determine failure metrics for target tissue exposed to vibration, using inputs of surrounding tissue, which are characterized as a system of mechanisms representative of a task, the method comprising:
(a) determining a tool and a risk factor associated with the task;
(b) collecting data from sensors that are monitoring behavior of the target tissue and the surrounding tissue while the tool is being used to perform the task, the sensors being positioned at pre-selected nodes and insertions in proximity to the target tissue and the surrounding tissue;
(c) repeating (a) and (b) for a plurality of tools, and a plurality of risk factors;
(d) determining a representative metric by comparing amplitude and frequency distributions of the collected data at the target tissue and the surrounding tissue;
(e) providing the representative metric to a finite element model (FEM);
(f) predicting and modeling effective resultant forces, frequencies, and amplitudes at a first location in the target tissue, for an applied force, vibration frequencies, and amplitudes experienced at a second location in the surrounding tissue for the tool and the risk factor;
(g) determining an FEM geometry for the first location in the target tissue based on the effective resultant forces, the vibration frequencies, and the amplitudes at the first location in the target tissue;
(h) determining material properties for the target tissue and the surrounding tissue;
(i) determining boundary conditions and static forces before use of the tool;
(j) providing the collected data, the FEM geometry, the material properties, the boundary conditions, and the static forces to the FEM; and
(k) determining, by executing the FEM, the failure metrics when the first location in the target tissue will fail for the plurality of the risk factors and the plurality of the tools.

2. The method of claim 1, wherein the sensors comprise:
accelerometers.

3. The method of claim 1, wherein the representative metric comprises:
an effective damping coefficient.

4. The method of claim 1, further comprising:
compiling the failure metrics; and
isolating a contribution of vibrations to the failure metrics from the contribution of the risk factor and a force exerted to operate the tool to determine a vibrating tool metric.

5. The method of claim 1, further comprising:
comparing failure predictions for the target tissue during usage of various tools used for the task and with the risk factor to determine a tool comparison metric.

6. The method of claim 5, further comprising:
assessing a likelihood of injury to the target tissue based on the tool comparison metric.

7. The method of claim 1, wherein the risk factor comprises:
posture, force, repetition, and vibration.

8. The method of claim 1, wherein the target tissue and the surrounding tissue comprise:
anatomical tissue.

9. The method of claim 1, wherein the pre-selected nodes comprise:
anatomical locations affected by the task.

10. The method of claim 1, further comprising:
providing inputs to the FEM including:
dimensional and material properties, including digital anatomical geometry, of the target tissue and the surrounding tissue;
static stress loading on the target tissue and the surrounding tissue based on a position of the target tissue and the surrounding tissue and a force of work to perform the task by the target tissue and surrounding tissue;
dynamic stress loading on the target tissue and the surrounding tissue based on tool; and
duration and repetition of the task.

11. The method of claim 10, wherein the dynamic stress comprises:
vibration.

12. The method of claim 10, wherein the static stress comprises:
a combination of a posture taken to operate the tool, the applied force exerted to operate the tool, and the repetition of the task.

13. A computer system operable to determine failure metrics for target tissue exposed to vibration, using inputs of surrounding tissue, which are characterized as a system of mechanisms representative of a task, the computer system comprising:
a hardware processor;
a non-volatile storage medium storing instructions that when executed by the hardware processor perform operations comprising:
(a) determining a tool and a risk factor associated with the task;
(b) collecting data from sensors that are monitoring behavior of the target tissue and the surrounding tissue while the tool is being used to perform the task, the sensors being positioned at pre-selected nodes and insertions in proximity to the target tissue and the surrounding tissue;
(c) repeating (a) and (b) for a plurality of tools, and a plurality of risk factors;
(d) determining a representative metric by comparing amplitude and frequency distributions of the collected data at the target tissue and the surrounding tissue;
(e) providing the representative metric to a finite element model (FEM);
(f) predicting and modeling effective resultant forces, frequencies, and amplitudes at a first location in the target tissue, for an applied force, vibration frequencies, and amplitudes experienced at a second location in the surrounding tissue for the tool and the risk factor;
(g) determining an FEM geometry for the first location in the target tissue based on the effective resultant forces, the vibration frequencies, and the amplitudes at the first location in the target tissue;
(h) determining material properties for the target tissue and the surrounding tissue;
(i) determining boundary conditions and static forces before use of the tool;
(j) providing the collected data, the FEM geometry, the material properties, the boundary conditions, and the static forces to the FEM; and
(k) determining, by executing the FEM, the failure metrics when the first location in the target tissue will fail for the plurality of the risk factors and the plurality of the tools.

14. The computer system of claim 13, wherein the sensors comprise:
accelerometers.

15. The computer system of claim 13, wherein the representative metric comprises:
an effective damping coefficient.

16. The computer system of claim 13, further comprising:
compiling the failure metrics; and
isolating a contribution of vibrations to the failure metrics from the contribution of the risk factor and force exerted to operate the tool to determine a vibrating tool metric.

17. The computer system of claim 13, further comprising:
comparing failure predictions for the target tissue during usage of various tools used for the task and with the risk factor to determine a tool comparison metric.

18. The computer system of claim 17, further comprising:
assessing a likelihood of injury to the target tissue based on the tool comparison metric.

19. The computer system of claim 13, wherein the risk factor comprises:
posture, force, repetition, and vibration.

20. A computer program product operable to determine failure metrics for target tissue exposed to vibration, using inputs of surrounding tissue, which are characterized as a system of mechanisms representative of a task, the computer program product comprising a computer readable storage medium having program instructions embodied therewith, the program instructions executable by a computing device to cause the computing device to perform operations comprising:
(a) determining a tool and a risk factor associated with the task;
(b) collecting data from sensors that are monitoring behavior of the target tissue and the surrounding tissue while the tool is being used to perform the task, the sensors being positioned at pre-selected nodes and insertions in proximity to the target tissue and the surrounding tissue;
(c) repeating (a) and (b) for a plurality of tools, and a plurality of risk factors;
(d) determining a representative metric by comparing amplitude and frequency distributions of the collected data at the target tissue and the surrounding tissue;
(e) providing the representative metric to a finite element model (FEM);
(f) predicting and modeling effective resultant forces, frequencies, and amplitudes at a first location in the target tissue, for an applied force, vibration frequencies, and amplitudes experienced at a second location in the surrounding tissue for the tool and the risk factor;
(g) determining an FEM geometry for the first location in the target tissue based on the effective resultant forces, the vibration frequencies, and the amplitudes at the first location in the target tissue;
(h) determining material properties for the target tissue and the surrounding tissue;
(i) determining boundary conditions and static forces before use of the tool;
(j) providing the collected data, the FEM geometry, the material properties, the boundary conditions, and the static forces to the FEM; and
(k) determining, by executing the FEM, the failure metrics when the first location in the target tissue will fail for the plurality of the risk factors and the plurality of the tools.
